(19) United States
(12) Patent Application Publication
(10) Pub. No.: US 2007/0232839 A1
(43) Pub. Date: Oct. 4, 2007

(54) NOVEL POLYNUCLEAR POLYPHENOL COMPOUND

(76) Inventors: Akira Yoshitomo, Wakayama (JP); Tatsuya Iwai, Wakayama (JP)

Correspondence Address: KNOBBE MARTENS OLSON & BEAR LLP, 2040 MAIN STREET, FOURTEENTH FLOOR, IRVINE, CA, 92614, US

(21) Appl. No.: 11/692,854

(22) Filed: Mar. 28, 2007

(30) Foreign Application Priority Data
Mar. 31, 2006 (JP) 2006-096341
May 11, 2006 (JP) 2006-132287
Mar. 16, 2007 (JP) 2007-067771

(51) Int. Cl. C07C 39/12

(52) U.S. Cl. 568/720

(57) ABSTRACT

A polynuclear polyphenol compound is constituted by, as a main skeleton, a 4,4′-methylene bis phenol structure where tris phenol skeletons of tris phenyl methane type are mutually bonded by methylene groups, wherein the reactivity of two hydroxyl groups bonded to the central skeleton of 4,4′-methylene bis phenol is significantly different from the reactivity of hydroxyl groups respectively bonded to the four phenyl groups of the two diphenyl methyl substituted groups.

### BACKGROUND OF THE INVENTION

1. Field of the Invention

The present invention relates to a new polynuclear polyphenol compound, and more specifically relates to a polynuclear polyphenol compound having a 4,4′-methylene bis(o-alkyl substituted phenol) structure where tris phenol skeletons of tris phenyl methane type are mutually bonded by methylene groups.

Such polynuclear polyphenol compound is useful in the production of base materials for photosensitive materials such as semiconductor photoresists, epoxy resin materials and hardeners used to seal integrated circuits, etc., agents to develop color or prevent fading used in thermosensitive recording materials, etc., as well as additives for bactericides, fungicides, etc.

2. Description of the Related Art

Polyphenol compounds of many different structures are known.

Among these polyphenol compounds, tris phenol compounds of tris phenyl methane type include, among others, 4,4′,4″-methylidyne tris phenol disclosed in Japanese Patent Laid-open No. Hei 6-115255, etc., 4,4′-[(4-hydroxyphenyl)methylene]bis [2-methyl phenol] disclosed in Japanese Patent Laid-open No. Hei 6-199717, etc., 4,4′-[(2-hydroxy phenyl)methylene] bis [2,3,5-trimethylphenol] disclosed in European Patent Application No. 510,672, and 4,4′-[(4-hydroxy phenyl)methylene] bis [2-cyclohexyl-5-methylphenol] and 4,4′[(2-hydroxyphenyl)methylene] bis [2-cyclohexyl-5-methylphenol] disclosed in Japanese Patent Laid-open No. Hei 6-1741.

Also, Japanese Patent Laid-open No. Hei 11-199533 discloses a polynuclear polyphenol compound mainly constituted by a 4,4′-methylene bis phenol structure where tris phenol skeletons of tris phenyl methane type are mutually bonded by methylene groups.
[Patent Literature 1] Japanese Patent Laid-open No. Hei 6-115255
[Patent Literature 2] Japanese Patent Laid-open No. Hei 6-199717
[Patent Literature 3] European Patent Application No. 510,672
[Patent Literature 4] Japanese Patent Laid-open No. Hei 6-1741
[Patent Literature 5] Japanese Patent Laid-open No. Hei 11-199533

These polynuclear polyphenol compounds themselves have many aromatic nucleuses and therefore exhibit excellent lipophilic property. If these aromatic nucleuses have alkyl groups or cycloalkyl groups as substitution groups, the above lipophilic property increases further. Also, presence of a large amount of phenol hydroxyl groups in the molecule also expands potential application fields of these polynuclear polyphenol compounds. For example, it is expected that these polynuclear polyphenol compounds can be used as materials for various types of resins offering improved properties such as water resistance, heat resistance and electrical characteristics.

They are also useful in the field of photosensitive materials such as photoresists, because resolution, development property and other performances required of photosensitive materials are determined by the reactivity with the chemical compound containing photosensitive groups or affinity with the development solution. As a result, polynuclear polyphenol compounds whose molecule contains many hydroxyl groups, where the reactivity of many hydroxyl groups in the polyphenol compound molecule varies at different levels, are being sought.

Among others, with polynuclear polyphenol compounds in which tris phenol skeletons of tris phenyl methane are mutually bonded by methylene groups, such as the aforementioned polynuclear polyphenol compound described in Japanese Patent Laid-open No. Hei 11-199533, the inventors confirmed that if an attempt is made to selectively substitute with protection groups, acid-decomposition groups, etc., those hydroxyl groups respectively bonded to the four phenyl groups of the two diphenyl methyl substituted groups among the six phenol hydroxyl groups contained in the polynuclear polyphenol compound, the intended selective substitution does not occur sufficiently. A likely cause is the small reactivity difference with respect to the hydroxyl groups bonded to the two phenyl groups in the 4,4′-methylene bis phenol structure, which is the central skeleton. This is one of the areas where polynuclear polyphenol compounds require further improvements.

### SUMMARY OF THE INVENTION

An object of the present invention is to provide a polynuclear polyphenol compound constituted by, as a main skeleton, a 4,4′-methylene bis phenol structure where tris phenol skeletons of tris phenyl methane type are mutually bonded by methylene groups, wherein the reactivity of two hydroxyl groups bonded to the central skeleton of 4,4′-methylene bis phenol is significantly different from the reactivity of hydroxyl groups respectively bonded to the four phenyl groups of the two diphenyl methyl substituted groups.

Having these two types of hydroxyl groups whose reactivity levels are significantly different, this polynuclear polyphenol compound may be useful, for example, in the application field of photosensitive materials such as photoresists. Specifically, when introducing protection groups, acid-decomposition groups, etc., such as t-butoxycarbonyl groups, to hydroxyl groups in the polynuclear polyphenol compound, these protection groups, acid-decomposition groups, etc., are preferentially introduced to the hydroxyl groups bonded to the four phenyl groups of the two diphenyl methyl substituted groups. It is expected that this will contribute to an improvement in photoresist resolution.

After examining ways to solve the aforementioned problems in earnest, the inventors found that, with respect to a polynuclear polyphenol compound constituted by, as a main skeleton, a 4,4′-methylene bis phenol structure where tris phenol skeletons of tris phenyl methane type are mutually bonded by methylene groups, introducing a lower alkyl group in the other o-position to the phenol hydroxyl groups bonded to the central skeleton of 4,4′-methylene bis phenol would allow for introduction of a diphenyl methyl substituted group in the aforementioned o-position in a quantitative manner equivalent to when no substitution group exists in the o-position, in addition to the diphenyl methyl substituted group substituted in one o-position. The inventors also found that this would also provide a polynuclear polyphenol compound serving the aforementioned object where the reactivity of phenol hydroxyl groups bonded to the 4,4′-methylene bis phenol skeleton is suppressed, as well as a method of synthesizing such chemical compound and the physical properties of the synthesized chemical compound. The present invention was developed based on these findings. It should also be added that this chemical compound has not heretofore been known.

To be specific, the new polynuclear polyphenol compound proposed by the present invention is expressed by the chemical formulas below.

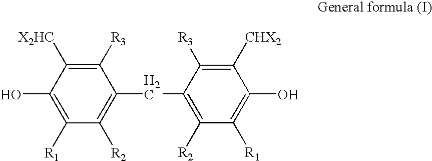

### (In the formula, R₁ represents an alkyl group of carbon number 1 to 4; R₂ and R₃ represent a hydrogen atom or alkyl group of carbon number 1 to 4; and X represents a hydroxy phenyl group expressed by general formula (II) specified below.)

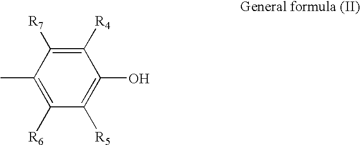

(In the formula, R₄ represents a hydrogen atom or methyl group; R₅ represents a hydrogen atom, alkyl group of carbon number 1 to 4 or cyclohexyl group; and R₆ and R₇ represent a hydrogen atom or alkyl group of carbon number 1 to 4. However, both R₆ and R₇ cannot be an alkyl group of carbon number 1 to 4 at the same time.)

In the above general formula (I), R₁ represents an alkyl group of carbon number 1 to 4. Specifically, alkyl groups of carbon number 1 to 4 include methyl groups, ethyl groups, propyl groups and butyl groups, among others. Propyl groups and butyl groups may be of straight-chain type or branched-chain type, but they should preferably be an alkyl group of carbon number 1 to 3, or more preferably a methyl group, ethyl group or isopropyl group. R₂ and R₃ represent a hydrogen atom or alkyl group of carbon number 1 to 4. Specifically, alkyl groups of carbon number 1 to 4 include those named as the candidate carbon-atom containing alkyl groups for R₁. With R₂ and R₃, however, preferably hydrogen atoms or methyl groups, or more preferably hydrogen atoms, should be used, because the methylene group at the center is not severed easily due to acid, etc., and thus the chemical structure remains stable.

As for the hydroxy phenyl group in the above general formula (II) represented by X, R₄ represents a hydrogen atom or methyl group, but it should preferably be a hydrogen atom. R₅ represents a hydrogen atom, alkyl group of carbon number 1 to 4 or cyclohexyl group. Specifically, alkyl groups of carbon number 1 to 4 include those named as the candidate carbon-atom containing alkyl groups for R₁. R₅ should preferably be an alkyl group of carbon number 1 to 3.

R₆ and R₇ represent a hydrogen atom or alkyl group of carbon number 1 to 4. However, both R₆ and R₇ cannot be an alkyl group of carbon number 1 to 4 at the same time. Specifically, alkyl group of carbon number 1 to 4 include those named as the candidate carbon-atom containing alkyl groups for R₁. R₆ and R₇ should preferably be a hydrogen atom or methyl group. In summary, desired modes of R₄ through R₇ are hydrogen atom for R₄, alkyl group of carbon number 1 to 3 for R₅, hydrogen atom for R₆, and methyl group for R₇.

### BRIEF DESCRIPTION OF THE DRAWINGS

FIG. 1a shows an HPLC analysis chart of the chemical compound obtained by the comparative applied example.

FIG. 1b shows an HPLC analysis chart of the chemical compound obtained by the applied example.

### DETAILED DESCRIPTION OF THE PREFERRED EMBODIMENT

The polynuclear polyphenol compound proposed by the present invention specifically includes, but is not limited to, any one of the following chemical compounds:

### 4,4′-methylene bis{2-[bis(4-hydroxy phenyl)methyl]-6-methylphenol} (Chemical Compound 1)

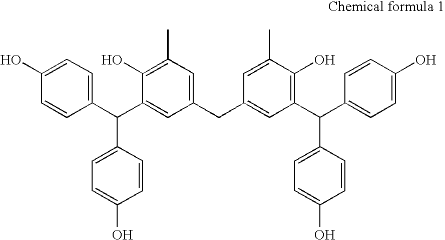

### 4,4′-methylene bis{2-[bis(3-methyl-4-hydroxyphenyl)methyl]-6-methylphenol} (Chemical Compound 2)

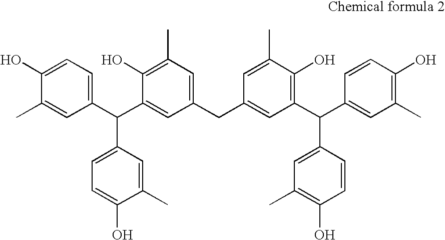

### 4,4′-methylene bis{2-[bis(2,5-dimethyl-4-hydroxyphenyl)methyl]-6-methylphenol} (Chemical Compound 3)

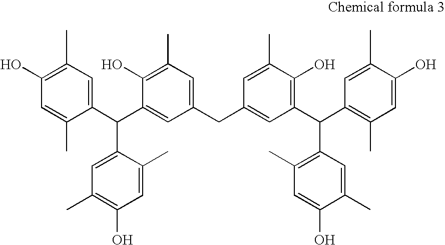

### 4,4′-methylene bis{2-[bis(2-methyl-5-cyclohexyl-4-hydroxyphenyl)methyl]-6-methylphenol} (Chemical Compound 4)

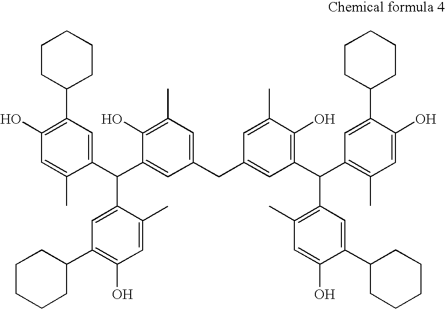

### 4,4′-methylene bis{2-[bis(2,3,5-trimethyl-4-hydroxyphenyl)methyl]-6-methylphenol} (Chemical Compound 5)

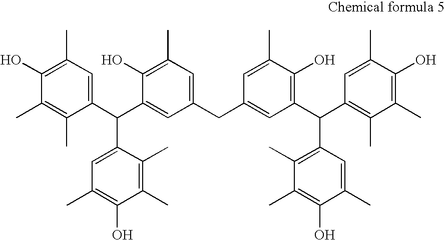

In the present disclosure, the word “the present invention” is intended to mean “an embodiment of the present invention” and should be understood accordingly.

The polynuclear polyphenol compound proposed by the present invention can also be any one of the following chemical compounds:
4,4′-methylene bis{2-[bis(2-methyl-5-ethyl-4-hydroxyphenyl)methyl]-6-methylphenol}
4,4′-methylene bis{2-[bis(2-methyl-5-isopropyl-4-hydroxyphenyl)methyl]-6-methylphenol}
4,4′-methylene bis{2-[bis(2,3-dimethyl-4-hydroxy phenyl)methyl]-6-methylphenol}
4,4′-methylene bis{2-[bis(2,5-dimethyl-4-hydroxy phenyl)methyl]-6-isopropylphenol}
4,4′-methylene bis{2-[bis(2-methyl-5-cyclohexyl-4-hydroxyphenyl)methyl]-6-isopropylphenol}
4,4′-methylene bis{2-[bis(2,5-dimethyl-4-hydroxy phenyl)methyl]-3,6-dimethylphenol}
4,4′-methylene bis{2-[bis(2-methyl-4-hydroxy phenyl)methyl]-6-methylphenol}
4,4′-methylene bis{2-[bis(3-methyl-4-hydroxy phenyl)methyl]-6-isopropylphenol}
4,4′-methylene bis{2-[bis(4-hydroxy phenyl)methyl]-6-isopropylphenol}

This new polynuclear polyphenol compound proposed by the present invention is not produced by any one specific method. As one viable industrial production method, however, it can be obtained by using, as a direct material, methylene bis(formyl phenol) that can be produced relatively efficiently from readily available materials such as alkyl hydroxy benzaldehydes, formaldehydes, etc., and reacting the material and phenol.

In other words, the new polynuclear polyphenol compound proposed by the present invention and expressed by general formula (I) can be obtained by reacting, in the presence of an acid catalyst, a methylene bis(formyl phenol) expressed by general formula (III) specified below and a phenol expressed by general formula (IV) specified below.

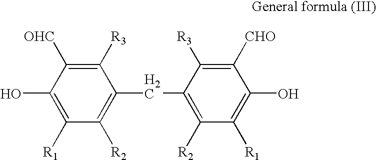

### (In the formula, R₁ through R₃ represent the same things as those in general formula (I).)

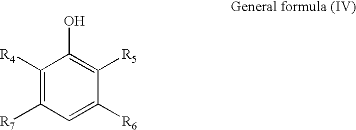

### (In the formula, R₄ through R₇ represent the same things as those in general formula (II).)

For example, a reaction formula used when the methylene bis(formyl phenol) expressed by general formula (III) is 4,4′-methylene bis(2-formyl-6-methylphenol) and the phenol expressed by general formula (IV) is 2,5-dimethyl phenol is shown below.

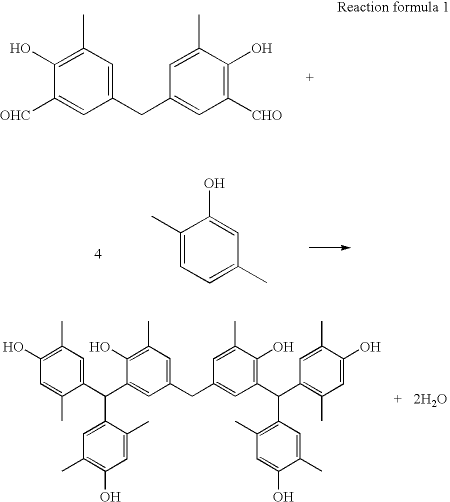

As for the methylene bis(formyl phenol) expressed by the above general formula (III), R₁, R₂ and R₃ represent the same things as those in general formula (I). With respect to the phenol expressed by the above general formula (IV), R₄, R₅, R₆ and R₇ represent the same things as those in general formula (II).

In other words, specifically the methylene bis(formyl phenol) expressed by the above general formula (III) is given as any one of the following chemical compounds, among others: 4,4′-methylene bis(2-formyl-6-methyl phenol), 4,4′-methylene bis(2-formyl-6-ethyl phenol), 4,4′-methylene bis(2-formyl-6-isopropyl phenol), 4,4′-methylene bis(2-formyl-3,6-dimethyl phenol), and 4,4′-methylene bis(2-formyl-5,6-dimethyl phenol).

This methylene bis(formyl phenol) can be produced from alkyl benzaldehyde and formaldehyde using the method described in “Chungnam Nation University Industrial Technology Development Center, Collection of Papers Vol. 4, No. 2, pp. 124 to 130 (1977), U.S. Pat. No. 2,775,613, or Journal of American Chemical Society, Vol. 79, pp. 6000 to 6002 (1957).”

To be specific, for example, the target product can be obtained by reacting an alkyl benzaldehyde expressed by general formula (V) with a formaldehyde, trioxane or other formaldehyde polymer using an acid catalyst or alkaline catalyst under a reaction condition where the formyl groups in the alkyl benzaldehyde do not react much, as shown in the reaction formula specified below. Here, in the case of carbonyl compounds other than formaldehydes, acetone, for example, does not react easily because the reactivity is lower than formaldehydes, and the benzaldehyde itself undergoes reaction under the conditions where acetone is reacted (specific temperatures, use of acid catalyst). For these and other reasons, aldehydes and ketones other than formaldehydes are not desirable.

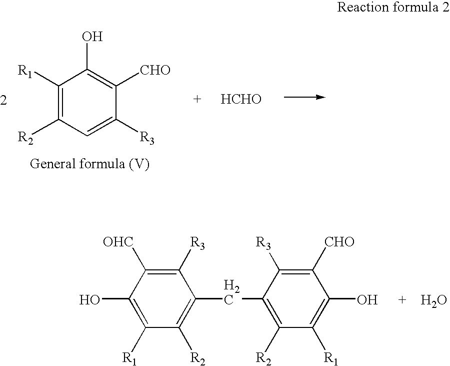

### (In the formula, R₁ through R₃ represent the same things as those in general formula (I).)

Specifically, the phenol expressed by general formula (IV) may be any one of the following chemical compounds, among others: Phenol, 2,5-xylenol, o-cresol, m-cresol, 2,3,6-trimethyl phenol, o-t-butyl phenol, 2-isopropyl phenol, 2-t-butyl-5-methyl phenol, 2-t-butyl-6-methyl phenol, 2-cyclohexyl phenol, 2-cyclohexyl-5-methyl phenol, and 2-sec-butyl phenol.

In the aforementioned reaction of methylene bis(formyl phenol) and phenol, at least 4 parts by mol, normally 4 to 15 parts by mol, or preferably 4.2 to 6 parts by mol, of phenol is used with respect to 1 part by mol of methylene bis(formyl phenol). Keeping the mol ratio too low is not desirable, because then 4 mols of phenol will not react with 1 mol of methylene bis(formyl phenol), thereby producing more byproduct and lowering the yield. As a result, the intended reaction product cannot be obtained at high purity.

In the aforementioned reaction of methylene bis(formyl phenol) and phenol, a reaction solvent may or may not be used. If a reaction solvent is used, it can be an aliphatic alcohol, aliphatic ketone, aromatic hydrocarbon, or any mixture thereof, in consideration of the solubility of the obtained product, reaction conditions, economy of reaction, and other aspects. To be specific, aliphatic alcohols that can be used as the reaction solvent include methanol, ethanol, isopropyl alcohol, n-propyl alcohol, t-butyl alcohol, isobutyl alcohol, n-butyl alcohol and other lower aliphatic alcohols, among others.

Aliphatic ketones that can be used as the reaction solvent include isopropyl ketone, methyl ethyl ketone, methyl isobutyl ketone and diisopropyl ketone, among others. Aromatic hydrocarbons that can be used as the reaction solvent include toluene, xylene and cumene, among others.

Among the candidates mentioned above, methanol, isopropanol and methyl isobutyl ketone can be used favorably as the reaction solvent.

These solvents are used by normally 10 to 500 parts by weight, or preferably 50 to 200 parts by weight, with respect to 100 parts by weight of the applicable phenol. However, the specific ratio of solvent is not limited to these ranges.

In producing the polynuclear polyphenol compound proposed by the present invention, the acid catalyst used in the reaction of methylene bis(formyl phenol) and phenol should preferably be an acid that dissolves in the reaction material or reaction solvent. In other words, favorable examples of this acid catalyst include hydrogen chloride, hydrochloric acid, sulfuric acid, sulfuric anhydrite, phosphoric acid and other inorganic acids; p-toluene sulfonic acid, methane sulfonic acid, trifluoromethane sulfonic acid and other organic sulfonic acids; oxalic acid, formic acid, trichloroacetic acid, trifluoroacetic acid and other carbonic acids; and cationic ion-exchange resins, among others. Among these, hydrogen chloride and hydrochloric acid are particularly preferable. Although the specific ratio varies depending on the concentration and type of the acid used, these acid catalysts are used normally by 5 to 50 percent by weight, or preferably 10 to 30 percent by weight, with respect to the phenol introduced.

The reaction should normally be implemented at a reaction temperature in a range of 0 to 100° C., or preferably in a range of 30 to 50° C., in air, or preferably in an atmosphere of nitrogen or other inactive gas, for around 2 to 48 hours, or normally 3 to 24 hours, under agitation. Under the present invention, the polynuclear polyphenol compound produced by this reaction normally contains various isomers and other byproducts in addition to the target product. If the target polynuclear polyphenol compound does not dissolve easily in the reaction solvent, normally the polynuclear polyphenol compound precipitates in the reaction solution as crystal at the aforementioned reaction temperature.

In this case, after the reaction is completed the obtained reacted mixture is mixed with ammonia water, aqueous sodium hydroxide solution or other aqueous alkaline solution to neutralize the acid catalyst, after which the reacted mixture is cooled, as necessary, and the precipitated coarse crystal is filtered. Next, this coarse crystal is dissolved in a solvent comprising aromatic hydrocarbon or aliphatic ketone or mixture thereof, and the obtained solution is washed with ion-exchanged water. Thereafter, the resulting solution is cooled to cause precipitation of crystal, and the crystal is filtered and dried. This way, the target polynuclear polyphenol compound can be obtained easily at high purity.

On the other hand, no crystal may precipitate in the reacted mixture after the reaction. In this case, after the reaction is completed the obtained reacted mixture is mixed with an alkali to neutralize the acid catalyst, as explained above, and thereafter a solvent that separates from water is added, as necessary, to separate and remove the water layer. The obtained organic layer is then distilled at normal pressure or reduced pressure, and the resulting distillation residue is dissolved by adding a solvent comprising aromatic hydrocarbon or aliphatic ketone or mixture thereof. The obtained solution is washed with ion-exchanged water and desalinated, and then the solvent is condensed to adjust the solvent content, as necessary, after which the adjusted solution is cooled to precipitate coarse crystal. Next, this coarse crystal is filtered and crystallized from the solvent comprising aromatic hydrocarbon or aliphatic ketone or mixture thereof. This way, the target polynuclear polyphenol compound can be obtained easily at high purity.

In the latter method, appropriate materials should be selected for the solvent used to dissolve the distillation residue, and the aforementioned solvent for crystallization, by considering the crystallization conditions, refining effect, economy, and the like. Examples of aromatic hydrocarbon include toluene, xylene and cumene, among others, while examples of ketone include isopropyl ketone, methyl ethyl ketone, methyl isobutyl ketone and diisopropyl ketone, among others. These crystallization solvents are added by normally 100 to 1,000 parts by weight, or preferably 200 to 500 parts by weight, with respect to 100 parts by weight of coarse crystal, to crystallize the target polynuclear polyphenol compound at high purity.

### Effects of the Invention

The new polynuclear polyphenol compound proposed by the present invention is a polynuclear polyphenol compound constituted by, as a main skeleton, the 4,4′-methylene bis phenol structure where tris phenol skeletons of tris phenol methane type are mutually bonded by methylene groups, wherein one molecule contains two hydroxyl groups bonded to the central skeleton of 4,4′-methylene bis phenol and four hydroxyl groups respectively bonded to the four phenyl groups of the two diphenyl methyl substituted groups, and wherein the reactivity levels of these two types of hydroxyl groups in the polynuclear polyphenol compound vary significantly. The difference in reactivity between the two types of hydroxyl groups can be checked by, for example, examining the composition distribution of the substitution product obtained by t-butoxycarbonyl substitution reaction. Having two types of hydroxyl groups of significantly different levels of reactivity, this polynuclear polyphenol compound conforming to the present invention can be used as a material in various reactions, such as reactions targeting phenol hydroxyl groups, as well as substitution reactions and hydrogenation reactions targeting phenol aromatic rings, to obtain a variety of derivatives not heretofore available.

For example, it is possible to selectively attach various substitution groups such as t-butylcarbonate or other protection groups or decomposition groups, to the hydroxyl groups positioned at the ends of the molecular structure in a manner bonded to the four phenyl groups of the two diphenyl methyl substituted groups in the polynuclear polyphenol compound proposed by the present invention. Therefore, use of the obtained substitution compound as a material or additive for photosensitive resist will likely improve the resolution. Also, introducing a methyl group to R₁ alone will significantly improve the melting point, which means that, when applied in the production of synthetic resin, the present invention can produce a synthetic resin offering not only improved heat resistance, but also improvements in other properties such as flexibility and water resistance.

In the present disclosure where conditions and/or structures are not specified, the skilled artisan in the art can readily provide such conditions and/or structures, in view of the present disclosure, as a matter of routine experimentation.

### Best Mode for Carrying Out the Present Invention

### EXAMPLE

### Example 1

### Synthesis of Chemical Compound 3

In a four-way flask with a capacity of 1 liter equipped with a thermometer, cooling equipment and agitator, 50.8 g (0.42 mol) of 2,5-xylenol and 50.8 g of methanol are introduced and then 39.5 g of hydrochloric acid gas is injected at a temperature of 30° C., after which a solution prepared by dissolving 101.7 g (0.83 mol) of 2,5-xylenol in 254.3 g of methanol was drip-fed under agitation. After the entire volume of the aforementioned solution was added, 71.0 g (0.25 mol) of 4,4′-methylene bis(2-formyl-6-methylphenol) was added to the obtained solution at 30° C. over a period of 2 hours, after which the mixture was agitated to cause reaction for 3 hours at 40° C.

After the reaction was completed, 16% aqueous sodium hydroxide solution was drip-fed to neutralize the reacted solution (crystal precipitated during the course of drip feed), after which methanol was added and the resulting solution was heated to 70° C. and then cooled and filtered to obtain coarse crystal. Next, this coarse crystal was transferred into a four-way flask with a capacity of 2 liters, together with methyl isobutyl ketone and water, and the mixture was heated to 70° C. to dissolve the crystal. Thereafter, the flask was kept stationary to remove the water layer, after which the obtained oil layer was mixed with water and thus washed in water in a similar manner to separate the oil layer. Next, the obtained oil layer was condensed at normal pressure and the solvent was distilled (crystal precipitated during the course of distillation), after which toluene was added. The resulting solution was cooled to 25° C., and the precipitated solid matter was filtered and dried to obtain 139.7 g of the target polynuclear polyphenol compound as powder of light yellow-white color, where the compound had a purity of 97.7% (as measured by high-performance liquid chromatography) and melting point of 307.0° C. (peak top value measured by differential scanning calorimetry, or DSC).

The yield with respect to 4,4′-methylene bis(2-formyl-6-methylphenol) was 75.8%.

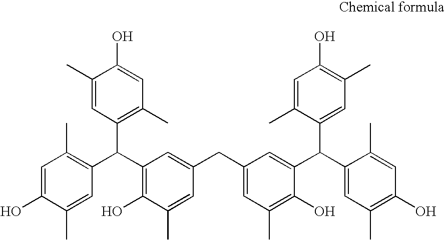

Molecular weight (as measured by mass spectrometry): 736 (M-H)⁻
Proton NMR analysis (400 MHz; solvent: DMSO-d6)

TABLE 1
Identification results by proton NMR
(Internal standard: tetramethyl silane)

| Shift value (ppm) | Number of protons | Signal | Assignment |
|---|---|---|---|
| 1.88–1.96 | 24 | m | —CH₃(2,5-Xylenol) |
| 2.08 | 6 | s | —CH₃ |
| 3.40 | 2 | s | —CH₂ |
| 5.70 | 2 | s | —CH |
| 6.31–6.33 | 6 | m | Ph-H |
| 6.51–6.54 | 6 | m | Ph-H |
| 7.91 | 2 | s | Ph-OH |
| 8.83 | 4 | s | Ph-OH(2,5-Xylenol) |

Applied example (Synthesis of t-butoxycarbonyl group substituted compound based on chemical compound 3):

In a 500-ml four-way flask, 7.4 g (0.1 mol) of chemical compound 3 obtained in accordance with Example 1 was introduced together with 22.2 g of methyl isobutyl ketone, and then 4.0 (0.4 mol) of triethyl amine was added. Next, the mixture was heated to 60° C., and 8.7 g (0.4 mol) of di-tert-butyl-dicarbonate was added over a period of 2 hours. Thereafter, the mixture was heated to 80° C. and then agitated to cause reaction for 4 hours.

After the reaction was completed, water was added to the reacted mixture at 60° C. to remove the water layer separated through the water-washing process. The remaining oil layer was then mixed with water again to repeat a similar operation, and the obtained oil layer was condensed, dried and solidified using an evaporator to obtain 11.8 g of yellow powder. The obtained powder was confirmed to be the target chemical compound based on NMR analysis. The chemical reaction formula below represents the operation in this applied example.

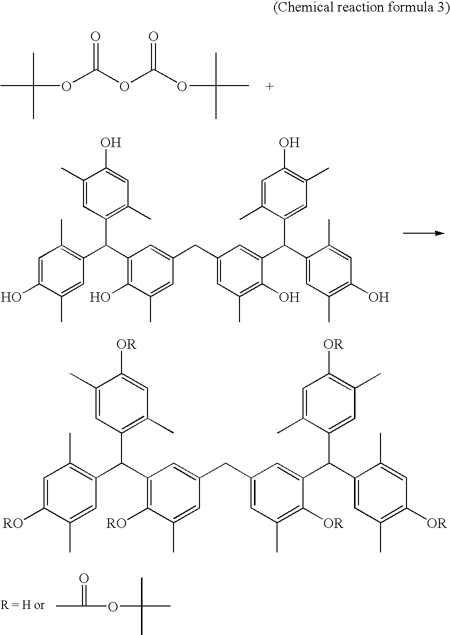

Comparative applied example (Synthesis of t-butoxycarbonyl group substituted compound based on 4,4′-methylene bis{2-[bis(2,5-dimethyl-4-hydroxy phenyl)methyl]-phenol}):

In a 500-ml four-way flask, 7.1 g (0.01 mol) of 4,4′-methylene bis{2-[bis(2,5-dimethyl-4-hydroxy phenyl)methyl]-phenol}, obtained from 2,5-xylenol and methylene bis salicylaldehyde in a manner similar to the method described in Japanese Patent Laid-open No. Hei 11-199533, was introduced together with 21.3 g of methyl isobutyl ketone, and then 4.0 g (0.04 mol) of triethyl amine was added. Next, the mixture was heated to 60° C., and 8.7 g (0.04 mol) of di-tert-butyl-dicarbonate was added over a period of 2 hours. Thereafter, the mixture was heated to 80° C. and then agitated to cause reaction for 4 hours.

After the reaction was completed, water was added to the reacted mixture at 60° C. to remove the water layer separated through the water-washing process. The remaining oil layer was then mixed with water again to repeat a similar operation, and the obtained oil layer was condensed, dried and solidified using an evaporator to obtain 11.3 g of yellow powder. The obtained powder was confirmed to be the target chemical compound based on NMR analysis. The chemical reaction formula below represents the operation in this comparative applied example.

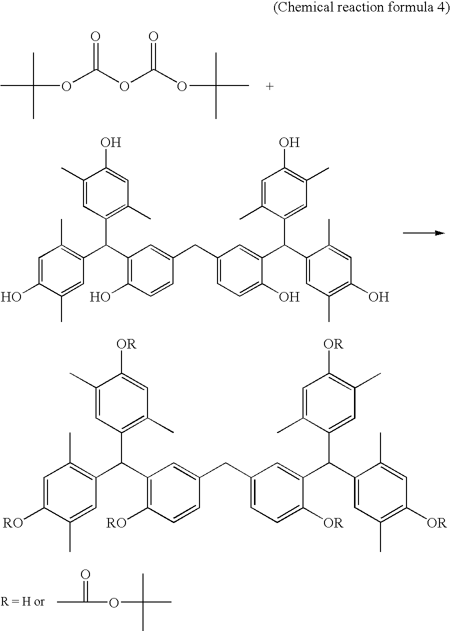

As shown by chemical reaction formulas 3 and 4, the chemical compounds obtained by the above applied example and comparative applied example were both a polynuclear polyphenol compound whose phenol hydroxyl groups were substituted by t-butylcarbonate groups, and also a mixture containing zero-position substitution product (unreacted product) up to six-position substitution product wherein all phenol hydroxyl groups were substituted. Accordingly, the composition value (area percentage on HPLC chromatograph) of each substitution product, from unreacted product to six-position substitution product, was obtained by high-performance liquid chromatography (HPLC) for the chemical compounds obtained by the applied example and comparative applied example. As a result, the chemical compound obtained by the applied example had very small percentages of one-position, five-position and six-position substitution products. Given the same number of substitution groups, the chemical compound obtained by the applied example had smaller numbers of peaks of 1% or above in composition ratio on the chromatograph, providing an evidence of selective reaction.

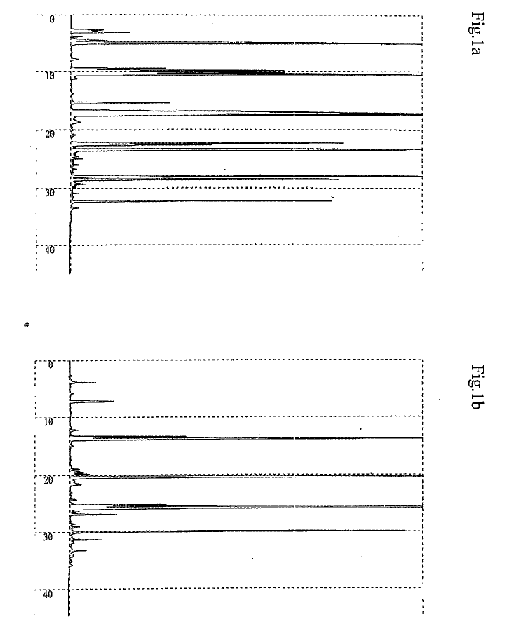

FIGS. 1a and 1b show the HPLC analysis charts.

Analysis conditions (Column: Shimpack CLC-ODS (manufactured by Shimadzu Corporation). Column temperature: 50° C. Detector: UV 280 nm. Development solvent and development method: Use a high-performance liquid chromatography method wherein development is started using an aqueous methanol solution comprising methanol and water mixed at a volume ratio of 80:20, and the concentration is changed to achieve a methanol content of 100 percent by volume 30 minutes later and development is performed for 30 minutes, and then upon elapse of 30 minutes development is performed for 15 more minutes using a development solvent with a methanol content of 100 percent by volume (analysis time: 45 minutes). Preparation method of measurement sample: 170 mg of reaction solution was diluted by methanol to prepare 50 ml of solution (containing approx. 50 mg of the target substance). Amount of sample introduced: 20 μL. Solvent flow rate: 1.0 ml/min.)

Table 2 lists the composition values (area percentages) obtained from FIGS. 1a and 1b.

TABLE 2
Composition distribution by number of substituted groups in substitution product (in area
percentage)

| | Chemical compound | | Chemical compound | |
|---|---|---|---|---|
| | obtained by | | obtained by | |
| | comparative | | applied | |
| | applied example | | example | |
| | | Number | | Number |
| | Total peak composition | of peaks | Total peak composition | of peaks |
| | value: Area percentage | of 1% or | value: Area percentage | of 1% or |
| Protection group | (hold time: minutes) | above | (hold time: minutes) | above |
| Unreacted | 2 (3.2) | 1 | 1 (3.9) | 1 |
| 1 position substituted | 9 (4.6~5.1) | 2 | 2 (7.4) | 1 |
| 2 positions substituted | 21 (9.5~10.6) | 4 | 15 (13.6~14.0) | 2 |
| 3 positions substituted | 27 (15.4~17.5) | 4 | 37 (19.1~20.5) | 1 |
| 4 positions substituted | 24 (22.3~23.5) | 3 | 40 (25.2~25.8) | 3 |
| 5 positions substituted | 13 (27.9~28.5) | 2 | 4 (30.0) | 1 |
| 6 positions substituted | 4 (32.3) | 1 | 4 (33.5) | 1 |

### (Each number of substitution groups in the substitution product is estimate, based on classification by peaks of similar hold time.)

Also for the chemical compound obtained by the applied example, the proton nuclear magnetic resonance method was used to measure the concentration of unreacted hydroxyl groups respectively bonded to the four phenyl groups of the two diphenyl methyl substituted groups, as well as the concentration of unreacted hydroxyl groups bonded to the central skeleton of 4,4′-methylene bis phenol. The results revealed that 87.8% of all hydroxyl groups bonded to the four phenyl groups of the diphenyl methyl groups were carbonated, while the percentage of carbonation among all two hydroxyl groups bonded to the central skeleton of 4,4′-methylene bis phenol was only 12.7%.

Although all possible variations are not listed herein, the present invention can be embodied in any modes incorporating various changes, modifications and improvements based on the knowledge of those skilled in the art. It goes without saying that these embodiments are also included in the scope of the present invention, as long as they do not deviate from the purpose of the present invention. Therefore, it should be clearly understood that the forms of the present invention are illustrative only and are not intended to limit the scope of the present invention.

The present application claims priority to Japanese Patent Application No. JP2006-096341, filed Mar. 31, 2006; Japanese Patent Application No. JP2006-132287, filed May 11, 2006; and Japanese Patent Application No. JP2007-067771, filed Mar. 16, 2007; the disclosure of which is incorporated herein by reference in their entirety.

What is claimed is:

1. A polynuclear polyphenol compound expressed by general formula (I): 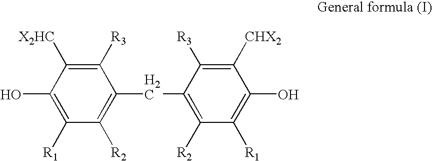
wherein R₁ represents an alkyl group of carbon number 1 to 4; R₂ and R₃ independently represent a hydrogen atom or alkyl group of carbon number 1 to 4; and each X independently represents a hydroxy phenyl group expressed by general formula (II), 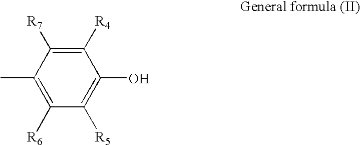
wherein R₄ represents a hydrogen atom or methyl group; R₅ represents a hydrogen atom, alkyl group of carbon number 1 to 4 or cyclohexyl group; and R₆ and R₇ independently represent a hydrogen atom or alkyl group of carbon number 1 to 4, where both R₆ and R₇ are not an alkyl group of carbon number 1 to 4 at the same time.

2. The compound according to claim 1, wherein R₁ represents an alkyl group of carbon number 1; R₂ and R₃ independently represent a hydrogen atom or alkyl group of carbon number 1.

3. The compound according to claim 1, wherein R₂ and R₃ represent a hydrogen atom.

4. The compound according to claim 2, wherein R₂ and R₃ represent a hydrogen atom.

5. The compound according to claim 1, wherein all of X represent the same hydroxy phenyl group.

6. A method for producing the polynuclear polyphenol compound of claim 1 comprising:
reacting a methylene bis(formyl phenol) expressed by general formula (III) and a phenol expressed by general formula (IV) in the presence of an acid catalyst, 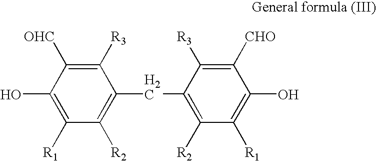
where R₁ represents an alkyl group of carbon number 1 to 4; R₂ and R₃ independently represent a hydrogen atom or alkyl group of carbon number 1 to 4, 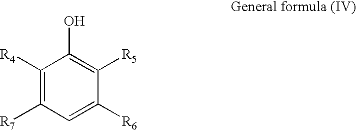
wherein R₄ represents a hydrogen atom or methyl group; R₅ represents a hydrogen atom, alkyl group of carbon number 1 to 4 or cyclohexyl group; and R₆ and R₇ independently represent a hydrogen atom or alkyl group of carbon number 1 to 4, where both R₆ and R₇ are not an alkyl group of carbon number 1 to 4 at the same time.
